(19) United States
(12) Patent Application Publication
(10) Pub. No.: US 2014/0025011 A1
(43) Pub. Date: Jan. 23, 2014

(54) SYSTEM AND METHOD FOR SUPPORTING A GASTRO-INTESTINAL TUBE

(71) Applicant: James J. Murtha, Burbank, CA (US)

(72) Inventor: James J. Murtha, Burbank, CA (US)

(21) Appl. No.: 13/552,503

(22) Filed: Jul. 18, 2012

(51) Int. Cl. A61M 39/10

(52) U.S. Cl. 604/180; 604/174

(57) ABSTRACT

A system for supporting a gastro-intestinal tube extending from a patient is described, the system comprising a pouch configured to receive the tube. The pouch has an interior and an exterior, the interior being configured to receive and hold a length of tubing. One or more fasteners are is mounted on a front side of the pouch for non-permanently attaching the pouch to a garment.

### BACKGROUND

The present invention relates to a securing device for medical tubes projecting from a person's body, and more particularly to a pouch-like securing device for medical tubes.

In modern medicine, numerous disorders may now be rectified by the insertion of medical tubes into the human body. As used herein, the term “medical tubes” refers to tubes which are used to bypass portions of the human body to aid certain bodily functions; examples of such tubes are catheters and gastro-intestinal tubes.

For example, and with reference to FIGS. 1-3, in the case of chronic reflux of food from the esophagus into the trachea or in patients where swallowing reflex has been compromised, some patients must be fed through a gastro-intestinal tube 100 inserted through a surgically-created hole which passes, initially, through the abdominal wall 102 and thence into the stomach 104. The purpose of such a tube is to provide a means for feeding individuals whose ailment prevents them from adequately feeding themselves. Individuals who might have extreme difficulty with feeding include individuals who have suffered neurological, or surgical damage, or damage related thereto, those who may suffer from deformity of the esophagus, or those who may have suffered an injury to the esophagus. Furthermore, a gastro-intestinal tube might be helpful for individuals who have had major surgery to the esophagus and are in a recovering condition.

With reference to FIG. 1, a gastro-intestinal tube 100 typically includes a flexible tube 106 having a primary entry port 108 and a secondary entry port 110 connected to a pair of corresponding exit ports at the opposite end (of which primary exit port 112 is seen in FIG. 1, the secondary port is not seen) via primary and secondary passages (also not seen in the figures). The primary entry port 108 is connected by a relatively large diameter primary passage to the primary exit port 112, and serves to allow food to enter the stomach. The secondary entry port 110 is connected by a relatively small diameter secondary passage to a balloon 114 positioned at the second exit port. This secondary passage serves to allow water to fill the balloon 114, and the balloon is configured, when inflated, to help to retain the gastro-intestinal tube 100 in place against the abdomen of the patient, as seen in FIG. 2. An adjustably slideable retainer member (or “bumper”) 116 is positioned to slide along the flexible tube 116, where it may be friction clipped into a desired position. By moving the retainer 116 up against the stomach 102 of the patient and clipped in that position, the balloon 114 and the retainer 116 apply opposing forces to the skin and stomach of the patient, thereby holding the gastro-intestinal tube 100 in a relatively stable position in relation to the patient.

In operation, the distal end of the gastro-intestinal tube is inserted, via a stoma in the abdomen wall, then through a gastric fistula into the person's stomach. The balloon 114 is filled by injecting sterile water through the secondary entry port 110. Preferably, once the gastro-intestinal tube is successfully inserted into the individual's stomach, and before the retainer member 116 is slipped into its final retention position, a suitable drainage sponge 40, which may preferably be a non-stick drainage sponge, is placed in position to surround both the stoma and the tube 100 protruding from the abdomen as seen in FIGS. 2-3. A suitable sponge for this purpose is provided by Dukal® Corporation, in the form of a “T-Drain Sponge” which is a square shaped sponge with a cut from one edge to the center, and with an opening in the center large enough to receive the diameter of the tube 100. In order to secure the sponge on the skin of the patient, an adhesive tape 50 having edges sufficiently large to protrude over the edges of the sponge, may be applied over the sponge to secure the sponge against the skin. In order to conveniently fit over the tube 100, the tape may also be cut from an edge to the center of the tape, in a configuration that substantially matches the cut in the sponge.

The gastro-intestinal tube 100 is then ready to be used to feed the person via the primary entry port 108. The tube may be used for a number of feedings before it has to be replaced. A portion of the tube, including the proximal end having the entry ports 108, 110 extends from the stoma in the abdominal wall. This portion of the gastro-intestinal tube is typically a source of considerable inconvenience for the person in whom the tube is placed, because it has a tendency to either slip into the stomach or to be pulled out of the stomach.

This kind of tube cannot be reused or re-sterilized once it has been removed from the stomach. Because they are relatively expensive, accidental or premature removal of a gastro-intestinal tube is not only a source of great discomfort for the user, it is also uneconomical. Thus, a number of approaches for reducing the chance of accidental or premature removal of gastronomy tubes have been developed.

In a first approach, a waist band, or a sling, may be provided for surrounding the torso of the patient, and a pocket or pouch is included in this garment configured for receiving the gastro-intestinal tube and holding it secure and immovable in relation to the patient's body. However, this approach has the disadvantage of imposing a tight fitting garment on the patient at the cost of physical discomfort because, in order to maintain the correct position of the pouch, the band cannot be loose fitting. Moreover, even a tight fitting waist band may be prone to slide downwardly when the patient moves.

In a second approach, specialized shirts or gowns have been developed with internal pouches or pockets permanently stitched or fused onto the internal surface of the garment, and located in a position suitable for receiving a gastro-intestinal tube. However, this approach has the disadvantage of imposing an expense and inconvenience on the user because each garment worn by the user requires specialized fabrication, and, each time the pocket must be cleaned, the entire garment must be cleaned.

It can thus be seen that there exists a need for an effective and economical means for holding a gastro-intestinal tube comfortably and which allows a patient freedom of movement, thereby reducing the chance of accidental or premature removal of the tube from the body of the patient. The present invention addresses these and other needs.

### SUMMARY OF THE INVENTION

In its most general aspect, the invention provides a pouch for holding the portion of a gastro-intestinal tube that extends from a user's body. The pouch is configured so that it can be removably affixed to a garment worn by the user. Such pouch allows the user to easily transfer the pouch between garments, eliminating the need for a dedicated garment having a pocket for holding the gastro-intestinal tube. The affixation method also allows the pouch to be positioned on the user's garment in such a way so that the user may optimize the location of the pouch for comfort while at rest, during exercise, and during the user's normal everyday activities.

In another aspect, the invention includes a system for supporting a gastro-intestinal tube extending from a patient, the system comprising: a pouch having an interior and an exterior, the interior configured to receive and hold a length of tubing; and a non-permanent fastener mounted on a front side of the pouch for non-permanently attaching the pouch to a garment. In one aspect, the pouch is formed from an anti-bacterial material. In another aspect, the anti-bacterial material is bamboo fabric.

In still another aspect, the pouch comprises two sheets of material stitched together. In yet another aspect, the pouch defines an aperture configured to receive the tube.

In still another aspect, the non-permanent fastener is two-sided adhesive tape. In another aspect, the two-sided adhesive tape does not require to be heated in order to set. In yet another aspect, the non-permanent fastener is a hook and loop fastener.

In a further aspect, the invention may include a garment to be worn by the patient, the garment configured to cover a location where the gastro-intestinal tube extends from the patient, wherein the pouch is removably attached to the garment at a location on the garment suitable for receiving and supporting the gastro-intestinal tube.

In still another aspect, the invention includes a kit for providing support for a gastro-intestinal tube extending from a stoma of a patient, the kit comprising: a pouch formed of a material, the pouch defining an aperture configured to receive the tube; a length of two-sided adhesive tape, configured so that one side is removably attachable to the pouch, the other side removably attachable to a garment to be worn by the patient; a sponge, configured to be placed against a portion of the patient's skin adjacent the stoma, and further configured to permit the gastro-intestinal tube to pass through the sponge; and an adhesive sheet configured to adhere the sponge to the patient's skin. In another aspect, the material has anti-bacterial properties. In still another aspect, the kit includes a pair of scissors. In yet another aspect, the adhesive sheet is formed from a silicone material.

In another aspect, the invention includes a method of supporting a portion of a gastro-intestinal tube that extends from the body of a patient, comprising: providing a pouch having an opening; removably attaching the pouch to a garment; and inserting a portion of the tube that extends from the patient through the opening into the pouch whereby the weight of the tube is supported by the pouch attached to the first garment.

In a further aspect, the invention includes removing the tube from the pouch; removing the pouch from the first garment; removably attaching the pouch to a second garment to be worn by the patient; and inserting the portion of the tube that extends from the patient through the opening into the pouch whereby the weight of the tube is supported by the pouch attached to the second garment.

Other features and advantages of the invention will become apparent from the following detailed description, taken in conjunction with the accompanying drawings, which illustrate, by way of example, the features of the invention.

### BRIEF DESCRIPTION OF THE DRAWINGS

FIG. 1 is a perspective view of a gastro-intestinal tube of the kind used in conjunction with the present invention.

FIG. 2 is a schematic drawing showing the anatomical configuration of how a gastro-intestinal tube in FIG. 1 may be used.

FIG. 3 is a schematic drawing showing a detail view of FIG. 2.

FIG. 4 is a front elevational view of a pouch having features of the present invention.

FIG. 5 is a rear elevational view of the pouch in FIG. 4.

FIG. 6 is a front elevational view of a garment used in conjunction with the pouch in FIG. 1

FIG. 7 is a view of items used as part of a kit including an embodiment of the invention.

### DETAILED DESCRIPTION OF THE PREFERRED EMBODIMENTS

The present application discloses embodiments of the invention, in which systems and methods are described for supporting a gastro-intestinal tube that extends from the body of a patient.

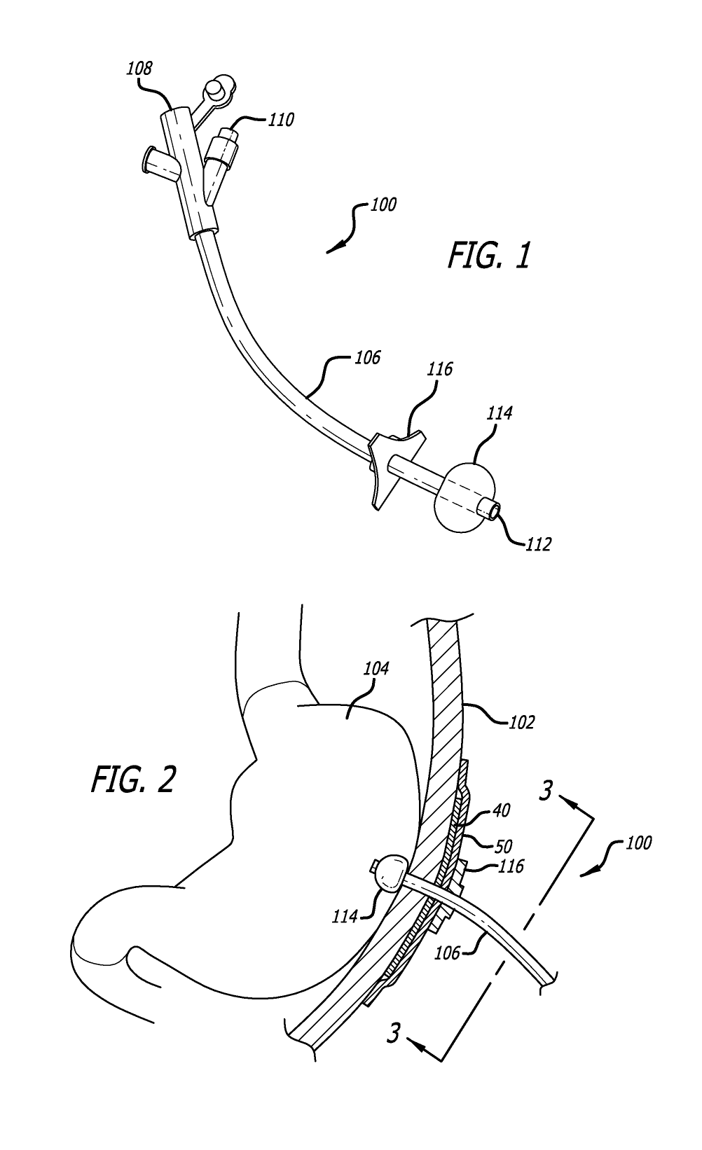

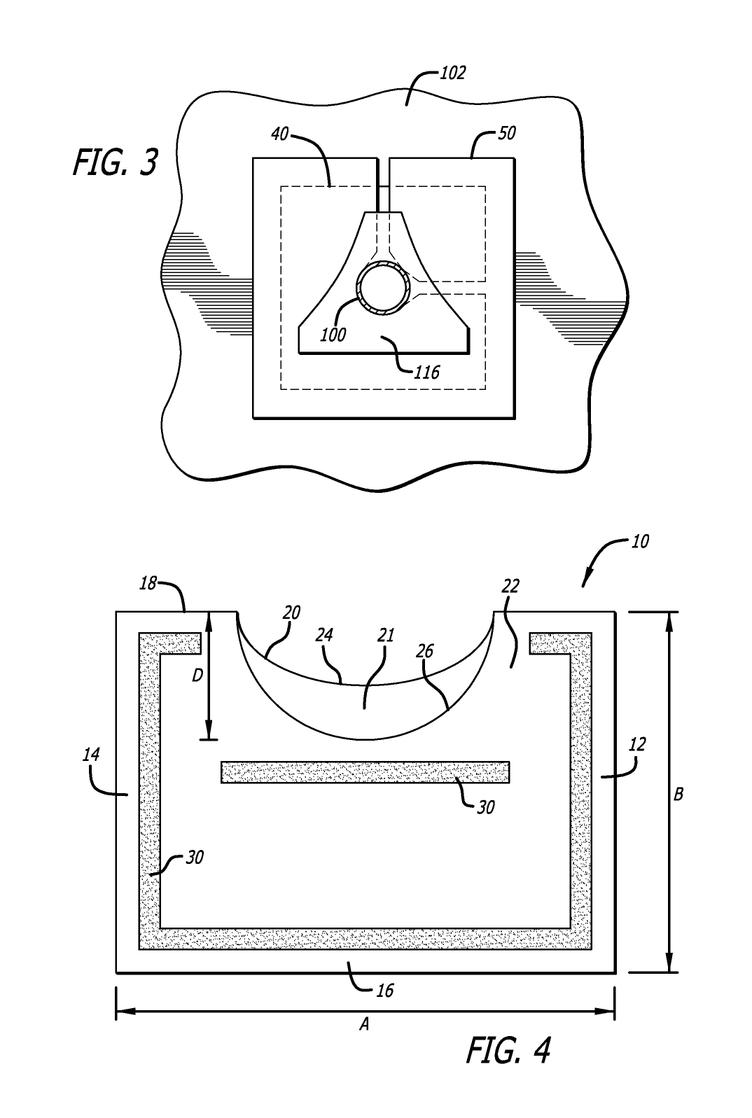

Included in the system and method of the present invention, FIG. 3 shows a front elevational view of an attachable and detachable pouch, generally identified by the numeral 10, configured for confining and supporting the weight of a gastro-intestinal tube 100 (also known as a gastrostomy tube) of the kind described above with reference to FIGS. 1-3.

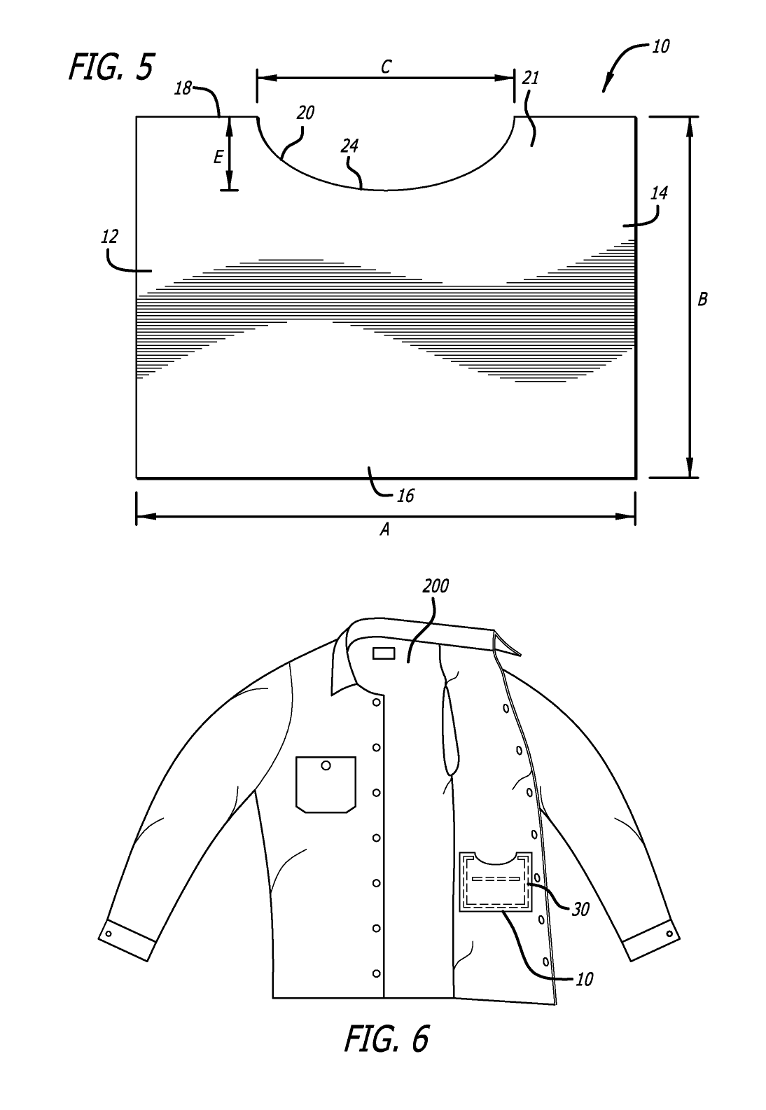

Referring now to FIG. 4, a pouch 10 comprises a generally rectangular shaped front sheet 22 and a back sheet 21, which are preferably stitched together along touching vertical edges 12, 14, and lower horizontal edge 16 and upper horizontal edge 18, using inturned ¼ inch seams so that a smooth edge presents itself to the user's skin. Preferably, the horizontal dimension “A” is between 6 inches and 12 inches, and the vertical dimension “B” is between 5 and 9 inches. The material is preferably an anti-bacterial bamboo fabric, which is breathable and washable, although the material may also be cotton or other synthetic material or the like. An opening 20 in the top of the pouch is provided, along a length of the upper horizontal edge 18, where the front and back sheet are not stitched together. At the location of the opening 20, the front sheet is cut so as to have a depression with a dimension “E” as shown in FIG. 5. The depression is configured to facilitate insertion of a gastro-intestinal tube 100 inside the pouch. Preferably, the dimension “E” is between 0.5 inches and 1.5 inches. Also at the location of the opening 20, the back sheet 21 is cut so as to have a depression with a dimension “D” as shown in FIG. 4. This depression is also configured to facilitate insertion of a gastro-intestinal tube 100 inside the pouch. Preferably, the dimension “D” is between 2 inches and 3 inches.

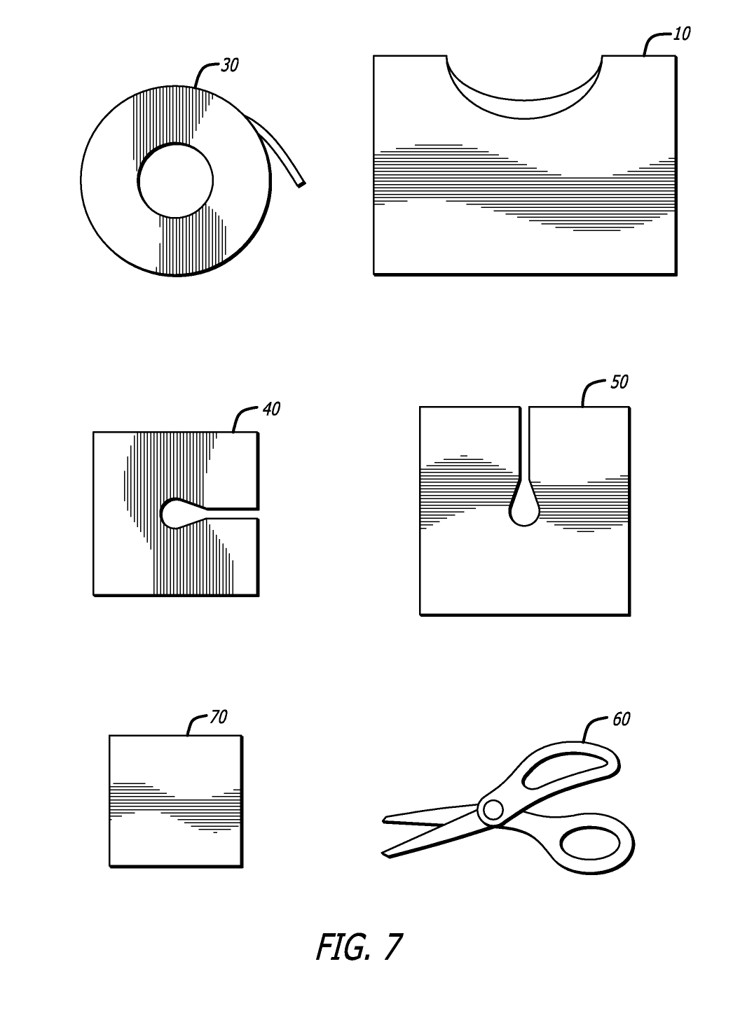

One advantage present in an embodiment of the invention, is that the pouch is not stitched or otherwise permanently attached to a garment to be worn by a patient. Rather, it is removably attached to the interior surface of a garment, preferably by means of a double sided instant bonding tape 30 as seen in FIG. 4. Preferably, the tape 30 does not require heat to set the adhesive bond. One product satisfying these preferred features is provided as an instant hem tape by Singer® which has sufficiently powerful adhesive qualities for the purpose of the invention. Another suitable product is a hook and loop type fastener such as made by Velcro®, or by 3M™. Additionally, ordinary adhesive tape or glue may be used. Where double sided tape 30 is used, it is attached on a first exposed side to the front sheet 22 of the pouch 10, preferably including around the perimeter of the pouch as seen in FIG. 4. A garment 200 to be worn by the patient is then prepared by exposing an internal surface of the garment near the abdominal area, and the pouch 10 with the adhesive tape in position is laid down on the internal surface so that the pouch 10 becomes temporarily attached to the garment, as seen in FIG. 6.

Once the pouch 10 is attached to the interior of the garment 200, the patient having a gastro-intestinal tube 100 extending from the abdomen may be dressed in the garment. The extending portion of the tube may be coiled, and may be easily inserted into the opening 20 provided in the pouch, where the weight of the tube will be suspended and secured thereby reducing any tendency for the weight of the tube to pull the balloon 114 out of the patient's stoma.

At any time that the patient decides that he or she would like to wear a different garment, the pouch 10 may be removed from the garment 200 by tearing apart the temporary bond supplied by the double sided tape 30, or by other forms of adhesive that may be used. After removing the double sided tape 30 completely from the pouch 10, fresh two-sided tape may be applied to the pouch and the pouch may be to the new garment for continued wear of the same pouch.

In some embodiments, the invention includes a kit of components that allows a patient who has been fitted with a gastro-intestinal tube to venture out in the world without fear of embarrassment and inconvenience. With reference to FIG. 7, elements that are included in one embodiment of a kit of the present invention are shown. Here, the kit comprises a pouch 10 with an opening positioned along an upper edge; a roll of double sided tape 30; a precut drainage sponge 40, wherein the cut is configured to receive a gastro-intestinal tube at the center of the sponge; an adhesive sheet which is dimensioned to adhere the drainage sponge onto the skin of a patient; and, a pair of scissors 60, suitable for cutting the adhesive tape from an edge towards the center thereof, the cut being configured to receive the gastro-intestinal tube at the center of the tape. While specific articles contained in the kit have been mentioned, one skilled in the art will immediately understand that substitutions for some or all of the various kit components, excluding the pouch, without departing from the scope of the intended invention. For example, double sides tape may be replaced by a different kind of fastener, such as a hook and loop fastener or the like. Similarly, the adhesive sheet may be formed from silicone, with an adhesive applied to one side of the silicone sheet, or the sheet may be formed from a naturally tacky silicone that is capable of adhering to the skin of a patient without any additional adhesive.

Thus, it will be appreciated, the system and method provided by the present invention provides a patient that uses the invention with a greater diversity of choice as to which garments the user would like to wear, at a greater degree of economy. For example, it will not be necessary to prepare beforehand each garment the user desires to wear by stitching a pouch onto the internal surface of the garment. It will not be necessary to wash an entire garment in the event that a pouch has become soiled or stained by fluid leaking from a stoma. Further, the kit of the present invention will provide a user with all the advantages of the novel pouch system described herein, and will also provide the advantage of mobility and ease of application to a user who is travelling or not hospitalized, and thus where professional treatment and equipment are not conveniently available. The present invention provides versatility, convenience, and economy, and successfully addresses problems found in the prior art.

While the present invention is described herein in terms of certain preferred embodiments, those skilled in the art will recognize that various modifications and improvements may be made to the invention without departing from the scope thereof. Moreover, although individual features of one embodiment of the invention may be discussed herein or shown in the drawings of the one embodiment and not in other embodiments, it should be apparent that individual features of one embodiment may be combined with one or more features of another embodiment or features from a plurality of embodiments.

I claim:

1. A system for supporting a gastro-intestinal tube extending from a patient, the system comprising:
a pouch having an interior and an exterior, the interior configured to receive and hold a length of tubing; and
a non-permanent fastener mounted on a front side of the pouch for non-permanently attaching the pouch to a garment.

2. The system of claim 1, wherein the pouch is formed from an anti-bacterial material.

3. The system of claim 2, wherein the anti-bacterial material is bamboo fabric.

4. The system of claim 1, wherein the pouch comprises two sheets of material stitched together.

5. The system of claim 1, wherein the pouch defines an aperture configured to receive the tube.

6. The system of claim 1, wherein the non-permanent fastener is two-sided adhesive tape.

7. The system of claim 6, wherein the two-sided adhesive tape does not require to be heated in order to set.

8. The system of claim 1, wherein the non-permanent fastener is a hook and loop fastener.

9. The system of claim 1, further including a garment to be worn by the patient, the garment configured to cover a location where the gastro-intestinal tube extends from the patient, wherein the pouch is removably attached to the garment at a location on the garment suitable for receiving and supporting the gastro-intestinal tube.

10. A kit for providing support for a gastro-intestinal tube extending from a stoma of a patient, the kit comprising:
a pouch formed of a material, the pouch defining an aperture configured to receive the tube;
a length of two-sided adhesive tape, configured so that one side is removably attachable to the pouch, the other side removably attachable to a garment to be worn by the patient;
a sponge, configured to be placed against a portion of the patient's skin adjacent the stoma, and further configured to permit the gastro-intestinal tube to pass through the sponge; and
an adhesive sheet configured to adhere the sponge to the patient's skin.

11. The kit of claim 10, wherein the material has anti-bacterial properties.

12. The kit of claim 10, further including a pair of scissors.

13. The kit of claim 10, wherein the adhesive sheet is formed from a silicone material.

14. A method of supporting a portion of a gastro-intestinal tube that extends from the body of a patient, comprising:
providing a pouch having an opening;
removably attaching the pouch to a garment; and
inserting a portion of the tube that extends from the patient through the opening into the pouch whereby the weight of the tube is supported by the pouch attached to the first garment.

15. The method of claim 14, further including:
removing the tube from the pouch;
removing the pouch from the first garment;
removably attaching the pouch to a second garment to be worn by the patient; and
inserting the portion of the tube that extends from the patient through the opening into the pouch whereby the weight of the tube is supported by the pouch attached to the second garment.
